(19) United States
(12) Patent Application Publication
(10) Pub. No.: US 2002/0137039 A1
(43) Pub. Date: Sep. 26, 2002

(54) 5' Nuclease nucleic acid amplification assay having an improved internal control

(75) Inventor: Matthias Gessner, Gross Enzersdorf (AT)

Correspondence Address: John P. Isacson, HELLER, EHRMAN, WHITE & McAULIFFE LLP, 1666 K Street NW, Suite 300, Washington,, DC, 20006-1228, US

(73) Assignee: Baxter Aktiengesellschaft

(21) Appl. No.: 09/746,874

(22) Filed: Dec. 22, 2000

(51) Int. Cl. C12Q 1/68; C12P 19/34

(52) U.S. Cl. 435/6; 435/91.2

(57) ABSTRACT

Nucleic acid amplification assays using a 5′ nuclease and having internal amplification controls are provided. Related methods for preparing the internal controls are also provided. Moreover, methods for rapidly and accurately determining optimum nucleic acid sequences for the internal amplification controls the 5′ nuclease assays are provided.

### FIELD OF THE INVENTION

The present invention provides 5′ nuclease assays having internal controls for detecting target nucleic acid sequences in samples. Specifically, the present invention provides improved methods for making and using internal controls for 5′ nuclease assays. More specifically the present invention provides methods for quickly and accurately determining optimum nucleic acid sequences for use as internal amplification controls in 5′ nuclease PCR assays.

### BACKGROUND OF THE INVENTION

Samples recovered from crime scenes, archeological diggings, environmental sites, and living organisms are often analyzed to determine what, if any, life forms are present. These samples can be analyzed using a variety of techniques including direct and microscopic examination, microbiological culturing, chemical analysis, immunoassays and nucleic acid detection. The assay's sensitivity and specificity is determined by the analytical method chosen, the sample's composition and quality and the nature of the analyte to be detected. Moreover, samples that contain only ancient life form remnants, traces of materials from complex higher organisms or dead and uncultivable microorganisms are especially vexing to analyze. Immunoassays using antibodies directed against a variety of antigens associated with suspected life forms can provide clues to the biological material's identity. Skilled microscopists can combine light and electron microscopy to screen samples for a wide range of possible life forms. Moreover, molecular biology techniques using labeled nucleic acid probes can be employed to identify specific target gene sequences. However, regardless of the analytical method chosen, analyte detection limits ultimately determine the assay's sensitivity.

An analytical technique's sensitivity is increased when analytes present in a sample are amplified. Amplification techniques include chemical extraction, affinity chromatography and microbial culturing, to name a few. However, each of these amplification techniques has significant limitations. Chemical extraction requires a basic knowledge of the chemical species sought and the nature of contaminating materials. Moreover, many compounds are too chemically similar to be separated and purified using extraction techniques. Furthermore, chemical analysis of biological samples is non-specific and precise identification of purified biological compounds is difficult. Affinity chromatography combined with immunoassay analysis has better specificity than chemical analysis alone, but is highly dependent on antibody selection. Microbiological culturing techniques can be exquisitely sensitive. However, these microbiological enrichment techniques require viable microorganism.

Early attempts to perform nucleic acid analysis using dot blot techniques and other in situ detection procedures (see for example Falkow et al. U.S. Pat. No. (U.S. Pat. No.) 4,358,535) demonstrated superb specificity but lacked sensitivity for many of the same reasons associated with the chemical, immunological and microbiological assays discussed above. However, in the 1980s nucleic acid amplification techniques we developed by Cetus Corporation researcher Kary Mullis (see U.S. Pat. Nos. 4,683,202, 4,683,195, 4,800,195 and 4,965,188; see also Saiki, R. K. et al. 1985. Enzymatic amplification of β-globin genomic sequences and restriction site analysis for the diagnosis of sickle-cell anemia Science 230:1350-1354). This Nobel Prize winning breakthrough in nucleic acid analysis made it possible to amplify trace amounts of nucleic acids by a factor of 10⁹. As a result, it was now possible to accurately detect and identify ancient life form remnants, traces materials from complex higher organisms and dead and uncultivable microorganisms with greater sensitivity and specificity than ever before.

Before proceeding further with the present background discussion, the following definition of terms is being provided as an aid to the reader. These definitions will be used throughout the remainder of this document. All other terms used are to be given their ordinary meaning as understood by those skilled in the art of molecular biology.

| | |
|---|---|
| oligonucleotide | A chain of more than one nucleic acid. |
| PCR master mix | A mixture containing all of the reagents necessary |
| | to perform a PCR assay except the test sample. A |
| | typical PCR cocktail will contain dNTPs, primers, |
| | probes, optionally internal standards, |
| | polymerase/endonuclease enzymes as well as |
| | buffers and cofactors. |
| primers | A pair of oligonucleotides specifically selected to |
| | hybridize at precise locations on complementary |
| | strands of nucleic acid present in the sample. The |
| | primers flank the target nucleic acid sequence and |
| | serve as initiation sites for the PCR assay. |
| probe | An oligonucleotide complementary to the target |
| | nucleic acid sequence. Used to detect the |
| | presence, or confirm the absence, of target nucleic |
| | acids in an amplified mixture. |
| reaction mixture | A mixture containing the test sample and PCR |
| | cocktail. |
| target nucleic acid | A nucleic acid sequence unique to the entity sought |
| sequence-(target | to be detected or identified. |
| oligonucleotide) | |
| test sample | A specimen to be tested using the assays described |
| | herein. |

The nucleic acid amplification technique developed by Dr. Mullis is called the polymerase chain reaction assay or “PCR” for short. Since PCR's advent numerous additional in vitro “amplification techniques” have been developed. Generally, there are three classes of nucleic acid amplification systems: (1) target amplification systems which use PCR, self-sustaining sequence replication (3SR) and strand displacement amplification (SDA); (2) probe amplification systems such as ligase chain reaction (LCR) and (3) signal amplification such as branched-probe technologies. Generally speaking, target amplification systems are preferred to other methods because the nucleic acid strand of interest is amplified making it available for sequence analysis, cloning and recombinant DNA applications. Therefore, PCR has remained the method of choice in most molecular biology laboratories worldwide.

Basically, PCR can be defined as an in vitro method for the enzymatic synthesis of specific DNA sequences using two oligonucleotide primers (probes) that hybridize to opposite strands and flank an area of interest in the target DNA. A repetitive series of reaction steps involving template denaturation, primer annealing the extension of the annealed primers by DNA polymerase results in the exponential accumulation of a specific target fragment whose termini are defined by the primers' 5′ ends. The PCR procedure uses repeated cycles of oligonucleotide-directed DNA synthesis to replication target nucleic acid sequences. In its most basic configuration, each PCR cycle consists of three discrete steps. The first step in PCR target nucleic acid amplification involves the addition of specific primers to a sample suspected of containing the target nucleic acid. The primers are designed to bind to complementary nucleic acid sequences present on opposite strands of DNA. The target nucleic acid sequence resides in-between the primer binding sights and is a unique marker characteristic of the agent to be detected. A cocktail containing the four desoxynucleoside triphosphates (dNTP), buffers containing magnesium salts, polymerase enzymes, and a variety of additives and cosolvents is mixed with the sample and primers. The PCR amplification process begins by denaturing DNA present in the sample using heat. The heat separates the DNA into two complementary strands. Next, the temperature is lowered to allow the primers, which have been added in molar excess, to bind (anneal) to their respective binding sights on the complementary strands of DNA. This is followed by primer extension where the primers are extended on the DNA template by a DNA polymerase. This cycle of denaturing, annealing and extension is repeated 40 to 50 times resulting in the exponential amplification of the target sequences.

PCR development has provided researchers with a reproducible, highly sensitive method for amplifying previously undetectable amounts of nucleic acid. However, detection of the amplified product requires additional sample manipulation. Initially, amplified nucleic acid sequences were detected using probes labeled with radioactive isotopes or conjugated to chromophores or enzymes. For example, the sample containing amplified product (or not) is spotted onto a solid substrate such as filter paper or a polymer membrane. Any nucleic acid present in the sample is then fixed to the substrate and reacted with a probe designed to hybridize with specific regions of the target nucleic acid sequence. Once hybridized, the labeled probe can be detected using methods appropriate for the label. Substrates having radioactively labeled probes hybridized to target nucleic acid sequences are exposed to x-ray film. If the radioactive probe has hybridized to the target nucleic acid (that is, if target nucleic acid is present in the amplified sample) the radioactivity of the label will leave an identifiable mark on the developed film. Samples lacking target nucleic acid will not hybridize with the probe and thus no radioactivity will be present and the developed x-ray film will remain blank. The most frequently used radioactive label is ³²P.

In another example, the probe is labeled with horseradish peroxides (HRP). After the probe has been allowed to hybridize with the target nucleic acid, the substrate is washed and a mixture containing tetramethylbenzidine (TMB) and peroxide is added. If the target nucleotide was present in the sample and hybridized with the HRP-labeled probe, the HRP will react with the peroxide in the TMB-peroxide mixture liberating reactive oxygen that then precipitates the TMB leaving a blue color on the substrate. In the absence of amplified target nucleic acid there will be no hybridized HRP-labeled probe present on the substrate, and hence nothing for the TMB-peroxide to react with. Consequently, the substrate remains colorless. There are many other examples of suitable post amplification detection systems that can be used with conventional PCR techniques. However, regardless of which post amplification identification system is used, considerable sample handling is required. As with any process, the more manipulation required the greater the opportunity for error introduction.

One technique for reducing post amplification processing provides a method of simultaneous target nucleic acid amplification and detection. This method relies on the 5′→3′ endonuclease activity of the DNA polymerase used in the primer extension step described above. A detailed example of a 5′→3′ endonucelase assay is provided in U.S. Pat. No. 5,210,015. During the primer extension step a DNA polymerase, such as but no limited to the thermophilc enzyme isolated from Thermus aquaticus and described in U.S. Pat. No. 4,889,818, is used to extend the primer. For example, when the target DNA is denatured it results in two complementary strands of DNA. Each complementary strand has a 5′ end and a 3′ end. Each single strand of DNA runs in the opposite direction of its complementary strand. The primers bind to their respective strands in the 5′→3′ direction. That is, primer extension always runs beginning at the 3′ end of the primer towards the 5′ end of the complementary strand to which it is bound. The DNA polymerase used in the assay moves along the complementary DNA strand from the 3′ end to the 5′ end. Each new nucleotide is added to the extending primer in the opposite orientation of the complementary target nucleotide strand so that the new nucleotides are orientated from 5′ to 3′ relative to themselves and the growing oligonucleotide primer. If oligonucleotides present in the reaction mixture bind to the target nucleotide strand ahead of the extending primer, the DNA polymerase will exert its 5′→3′ endonuclease activity and cleave the bound oligonucleotide.

The 5′→3′ endonuclease activity of DNA polymerase enzymes have been used to develop a method for the simultaneous PCR amplification and detection of target nucleic acid sequences. This assay, referred to herein after as the 5′ nuclease assay and known commercially as Taqman® (Roche Molecular Systems, Inc., Branchburg Township, N.J.) is generally performed as follows. Oligonucleotide probes are designed to bind to target nucleic acid sequences upstream of the extending primer. Each oligonucleotide probe is labeled at the 5′-end with a reporter molecule such as a fluorochrome and a reporter molecule quencher at the 3′ end (labeled probes). The labeled probes are added to the PCR reaction mixture along with the primer cocktail and sample. After the denaturing step, the reaction mixture is cooled to a point that favors the binding of the labeled probes preferentially to the primers. Next the reaction temperature is lowered to the optimum temperature for primer annealing and extension. As the DNA polymerase moves along the target nucleic acid strand from the 3′ end towards the 5′ adding dNTPs to the growing primer it will encounter the 5′ ends of the labeled probes previously bound to the target nucleic acid strand. When the DNA polymerase encounters these bound labeled probes it will exert its 5′→3′ endonuclease activity liberating these previously bound, labeled probes one nucleotide at a time into the reaction mixture.

The Taqman® assay is designed so that it will not detect reporter molecules that remain within a predetermined proximity of the quencher molecule. For example, a fluorescent molecule is conjugated to the 5′ end of a 10 nucleotide long probe that has a fluorescent quencher molecule bound to the 3′ end. The probe is complementary to a sequence found in the target nucleic acid sequence. When the PCR cocktail containing the polymerase, dNTP, primers and labeled probes are added to the sample forming a reaction mixture, the detection system does not recognize a fluorescent signal because the probe's fluorescent reported is quenched by the reporter quencher. However, during the PCR process the labeled probe will bind to target nucleic acid present in the reaction mixture. As the primers are extended in the 5′→3′ direction the polymerase will encounter labeled primer bound to the target nucleic acid downstream from the extending primer. As this occurs, the polymerase will exert its 5′nuclease activity and liberate the nucleotides of the labeled probe, either individually, or in small oligonucleotides. Consequently, the fluorescently labeled 5′ nucleic acid will be separated from the olignucleotide having the fluorescent quencher conjugated to its 3′end. Once liberated the fluorescent label is no longer quenched and can be detected by the a fluorometer or other suitable means. Unbound labeled probe present in the reaction mixture does not interfere with the assays because it remains quenched. Similarly, labeled probe non-specifically bound to nucleic acid sequences unrelated to the target nucleic acid will remain bound and quenched. Therefore, any free reporter detected in the sample mixture is directly proportional to the amount of labeled probe originally specifically bound, and hence target nucleic acid.

The PCR technique was quickly integrated into clinical laboratories due to its high level of sensitivity and specificity. However, the traditional PCR techniques were extremely labor intensive and highly susceptible to human error due the number of post amplification manipulations required to obtain a result. Consequently, numerous samples had to be repeated which even further increased workloads for the clinical laboratory staffs. Furthermore, the level of sophistication associated with PCR amplification and detection techniques required laboratories to hire and train experienced clinical scientists thus increasing labor costs considerably. However, with the advent of the 5′ nuclease assay, specifically the Taqmano technique, PCR assays became automatable thus allowing for significant reductions in labor and reagent costs.

The Taqman® assay made it possible to detect positive PCR reactions quickly and accurately for the first time. However, PCR generally, like many laboratory assays, can be prone to false negative results. That is, the target nucleic acid may be present in a sample but fails to be amplified for one reason or another. PCR is particularly prone to the adverse effects of inhibitors, many of which are commonly associated with samples of biological origin. Generally, a PCR inhibitor is any compound that inhibits the activity or the polymerase enzyme. Specific examples include heme and its metabolic products, acidic polysaccharides, detergents and chaotropic agents. Blood is a commonly used clinical sample and therefore the possibility of heme contamination cannot be ruled out. Moreover, many biological products are made from human plasma and serum.

It is well known that several of the most deadly infectious agents including human immunodeficiency virus (HIV), hepatitis type B virus and hepatitis type C virus are transmitted through contact with infected blood and blood products. The exquisite sensitivity and specificity of PCR makes it ideally suited for the detection of blood borne infectious agents. However, if PCR results are to be relied upon it is imperative that negative results be true negatives and not false negatives that result form assay failure.

Confidence in PCR assay negative results can be significantly increased when internal controls designed to confirm amplification and result integrity are integrated into the assay. An internal control can be added to the assay along with the PCR master mix described above, but the internal standard can be added to the sample prior to any possible pre-purification or extraction of the nucleic acid from the sample, as a result, false negative results which can arise from errors or losses from such pre-treatments can be filtered out. For example, in a 5′nuclease PCR assay designed to detect HIV, a primer pair directed against the group antigen (gag) region of the virus gene is constructed. Next a labeled probe having a sequence know to be complementary to a region of the HIV gag gene flanked by the primers is made. This combination of HIV gag specific primers and probes will be referred to as the “test detection system.” An internal control is then made. Generally, a synthetic oligonucleotide construct is prepared that contains a nucleic acid sequence different than the target region of the test detection system (the “internal control target”). Primer binding regions identical to the test detection system flank this internal control target. A labeled probe complementary to the internal control target is then provided to complete the internal control.

When the 5′nuclease PCR assay containing the internal control is performed false negatives will be easily detected by the absence of any signal. True negative samples will generate signal derived from the internal control system, but not from the test detection system. However, designing the internal control system can be a complex and vexing challenge. In order for an internal control in any assay system to be valid, there must be a minimum number of variables. That is, the internal control must mimic the test system as closely as possible. In the case of nucleic acid detection systems this problem is compounded by the variability associated with a probe's avidity for its complementary oligonucleotide sequence. Subtle differences in nucleic acid sequence can have profound effects of melting temperatures, annealing temperatures and nuclease activity. Consequently, present methods require an often-exhausting effort to design, test and ultimately develop reliable internal control systems. Internal control systems that have chemical properties that differ significantly form the test detection system's can lead to additional false negative results, and ever worse, a false sense of security in the assay's integrity.

Therefore, it is an object of the present invention to provide a method for developing 5′ nuclease assay internal control systems that closely mimic the assay test detection system.

It is another object of the present invention to provide 5′ nuclease assay internal control systems that can be designed easily and with a minimum amount of calculation, experimentation and development time.

It is yet another object of the present invention to provide a 5′ nuclease assay having internal control systems exactly mimicking test detect systems' avidity, specificity and sensitivity.

### BRIEF SUMMARY OF THE INVENTION

The 5′ nuclease assays of the present invention achieve these and other objects by incorporating an internal control into the PCR assay having a probe binding site that is the inverse of the target oligonucleotide probe binding site. Consequently, internal control/probe pairs can be designed for 5′ nuclease assays that have annealing properties and melting points nearly identical the target oligonucleotide/probe pairs without complex and tedious calculations.

In one embodiment of the present invention a method for preparing an internal control for a 5′ nuclease polymerase chain reaction (PCR) assay is provided. The method includes determining a nucleic acid sequence for a target oligonucleotide probe binding site and then inverting the nucleic acid sequence of the target oligonucleotide probe-binding site. Next an internal control oligonucleotide is constructed that contains the inverted target oligonucleotide probe binding site. The resulting internal control probe hybridizes with the inverted target nucleic acid probe binding site sequence integrated into the internal oligonucleotide control, but not with the nucleic acid sequence of the target oligonucleotide probe binding site.

In another embodiment of the present invention the internal control probes have detectable labels including, but not limited to, fluorescent labels, radioactive labels, antibody labels, chemiluminescent labels, paramagnetic labels, enzymes and enzyme substrates. In another embodiment both the internal control probes and the target oligonucleotide probes have detectable labels. In yet another embodiment of the present invention the target oligonucleotide detectable label is different than the internal control probe detectable label.

Another embodiment of the present invention consists of a 5′ nuclease PCR assay having an internal control where at least part of its nucleic acid sequence is the inverse of a target oligonucleotide probe binding site nucleic acid sequence. The assay also consists of an internal control probe having a nucleic acid sequence complementary to the inverted target oligonucleotide probe binding site where the internal control probe hybridizes with the inverted target nucleic acid probe binding site sequence but not with the target oligonucleotide probe binding site. The same primers amplify the internal control and target oligonucleotide of the present invention and the target oligonucleotide probe and internal control probe have different detectable labels.

In another embodiment of the present invention, the 5′ nuclease PCR assays are intended for the detection of pathogens including, but not limited to, human immunodeficiency viruses (HIV), hepatitis C virus (HCV), hepatitis B virus (HBV), human parvovirus, hepatitis A virus, alpha viruses, non-HIV retroviruses, enteroviruses, and non-viral pathogens.

The present invention also includes a quantitative HCV 5′ nuclease PCR assay having a labeled probe with a nucleic acid sequence complementary to a portion of an HCV oligonucleotide. The HCV oligonucleotide having primer binding sites. The primers are extendable by a 5′ nuclease enzyme. The assay also has an internal control oligonucleotide that has the same primer binding sites as the HCV oligonucleotide and an internal control probe binding site having a nucleic acid sequence that is the inverse of the HCV oligonucleotide probe binding site.

The probes used in the quantitative HCV 5′ nuclease PCR assay of the present invention are sufficiently different from each other to permit their individual detection. Suitable non-limiting probe label examples include, but are not limited to, fluorescent labels, radioactive labels, antibody labels, chemiluminescent labels, paramagnetic labels, enzymes and enzyme substrates.

Further objects and advantages of the 5′ nuclease PCR assays produced in accordance with the teachings of the present invention as well as a better understanding thereof, will be afforded to those skilled in the art from a consideration of the following detailed explanation of preferred exemplary embodiments thereof.

### BRIEF DESCRIPTION OF THE DRAWINGS

FIG. 1 depicts a method of using the wild type HCV plasmid pCK1 to prepare the internal control plasmid pCM1 having an inverted HCV wild type probe binding site in accordance with the teachings of the present invention.

### DETAILED DESCRIPTION OF THE INVENTION

Generally, the terms used to describe the present invention shall be given their ordinary meaning as known to those skilled in the art of molecular biology. However, the following terms will be further defined for the convenience of the reader. Oligonucleotide shall mean a molecule composed of more than one nucleic acid. Each nucleic acid shall be bound to one another via a phosphodiester bond between the 5′ end of one nucleic acid to the 3′ end of the other. Target oligonucleotide is the substrate the amplification assay of the present invention has been designed to detect. For example, a hepatitis C virus (HCV) polymerase chain reaction (PCR) assay is designed to detect HCV through a process including oligonucleotide amplification. The HCV oligonucleotide is the target oligonucleotide in an HCV PCR assay. Probe, or nucleic acid probe is an olignucleotide complementary to a specific region of the target oligonucleotide or internal control oligonucleotide. A labeled probe is a probe having a detectable compound attached thereto. Primer refers to a pair of oligonucleotides complementary to specific regions on individual strands of the target oligonucleotide or internal control oligonucleotide. The primers serve as amplification initiation sites and are extended through the action of the polymerase enzymes of the present invention. Internal control shall mean an oligonucleotide/probe pair that is discrete from the target oligonucleotide/probe pair. The internal standard of the present invention is intended to provide verification that the amplification assay worked as intended. Endonuclease, nuclease, 5′→3′ nuclease and 5′ nuclease are enzymes that cleave oligonucleotides, generally one nucleotide at a time, from their complementary oligonucleotide in the 5′→3′ direction. The terms 5′ and 3′ end refer to specific orientations of nucleic acids relative to an oligonucleotide molecule. 5′ nuclease assay and 5′ nuclease PCR assay shall mean a nucleic acid amplification assay that utilizes a polymerase enzyme for primer extension that also possess 5′→3′ nuclease activity.

The present invention provides a 5′ nuclease assay that utilizes an internal standard having molecular and chemical characteristics identical, or nearly identical to the target oligonucleotide/probe pair. Nucleic acid amplification assays including, but not limited to PCR, reverse transcriptase (RT) PCR, and Multi-plex PCR (referred to herein after collectively as “PCR”) have become one of the most commonly used and versatile molecular and diagnostic techniques used today. Virtually all research institutions and clinical laboratories use some form of PCR assay routinely.

Polymerase chain reaction assays are particularly well suited for screening samples of biological origin for infectious agents. Clinical specimens such as blood, tissue, semen, saliva tears and cerebral spinal fluid have the potential to transmit infectious agents such as, but not limited to human immunodeficiency virus (HIV), hepatitis C virus (HCV), hepatitis B virus (HBV), parvovirus B19, and human T cell lymphotropic viruses types I and II (collectively referred to herein after as blood borne pathogens). These blood borne pathogens cannot be easily detected using standard laboratory techniques such as virus cultures. Moreover, there is often a significant time lag between the time a person is exposed to a blood borne pathogen and the development of detectable antibodies (seroconversion). Many blood borne pathogens are highly infectious and cause debilitating diseases. Consequently it is imperative that clinical specimens as well as biological materials such as plasma and blood intended for in vivo use be tested using highly sensitive and specific techniques such as PCR.

When a blood borne pathogen is detected in a clinical specimen or other biological material the sample is retested to confirm the positive result. If the positive result is confirmed appropriate measures are taken. For example, if HCV is detected in a patient's serum, the patient is informed and any required therapy is initiated. When the sample is blood donated in vivo use, an HCV positive unit is destroyed to prevent transmission of the agent to an un-expecting blood product recipient. However, when a clinical specimen or other biological material is determined to be blood borne pathogen-free based on a negative PCR result, retesting is generally not performed. The patient is then informed of his negative status and donated blood is processed for in vivo use.

Generally, most analytical assays are performed using external controls designed to indicate whether the assay is performing properly. For example, an antibody detection immunoassay includes positive, negative and other assay controls that are run in parallel with clinical specimens. If the positive or negative results are out of their expected range, the assay is invalidated and repeated until the controls work properly. This type of assay control is referred to as an external control because they are run independently of the samples themselves. For most robust assays external controls are adequate to assure result integrity. However, nucleic acid amplification assays are extremely sample dependent assays. If trace amounts of polymerase enzyme inhibitors contaminate a sample, the assay will not work. All of the external assay controls will appear normal and a spectrum of positive and negative results will be recorded for that assay run. There is no way to determine if the negative results (no amplified target olignucleotide is present in the reaction mixture) are a true reflection that target oligoncleotide was absent, or if sample contamination inhibited the assay. Consequently, internal controls were developed that individually determine whether suitable amplification conditions existed in each sample when the PCR assay was conducted.

Internal PCR standards are generally composed of non-target oligonucleotides having nucleic acid sequences complementary to the assay primers. The internal control can be added to the sample along with a PCR master mixl that includes the polymerase enzyme, primers, buffers, cofactors, salts and other reagents appropriate for the assay being performed. After the amplification process is complete each sample is tested to determine whether target oligonucleotide andlor the internal standard oligonucleotide was amplified. Samples having both oligonucleotides amplified are considered positive, samples having only the internal control oligonucleotide amplified are considered confirmed negative. Samples in which neither target nor internal control oligonucleotide were amplified are considered false negatives. All false negative samples are then repeated.

Internal controls used in PCR assays must exhibit amplification properties and susceptibility to inhibitors that are approximately equal to target oligonucleotides. Internal standards that do not behave nearly identically to the target oligonucleotide in the PCR assay may further exacerbate problems with false positive and false negative results. For example, if an internal control is used that is significantly less sensitive to an inhibitor than the target oligonucleotide, it is possible that target oligonucleotide amplification will be inhibited, and not internal control oligonucleotide amplification. In this case, a false negative may be reported based on detection of internal control amplification in the assay. Designing suitable internal standards can be extremely demanding and technical challenging. This is especially true for the more complex nucleic acid amplification systems such as the 5′ nuclease assay.

The 5′ nuclease nucleic acid amplification assay utilizes polymerase enzymes that also exhibit endonuclease activity. The polymerase enzyme most commonly used in PCR assays, including 5′ nuclease assays, is Taq polymerase. Taq polymerase was originally isolated from the thermophilic bacteria Thermus aquaticus and exhibits 5′→3′ nuclease activity. In the 5′ nuclease assay internal controls can be added that validate assay result integrity. Briefly, synthetic oligonucleotides incorporating the same primer binding sites found in the target oligonucleotide are provided using molecular biology techniques known to those of ordinary skill in the art. However, the oligonucleotide sequence downstream of the primer binding sites (moving from the 3′ end towards the 5′ end of the strand) is different from the target oligonucleotide sequence. Probes are then provided that bind to either the target oligonucleotide sequence or the internal control oligonucleotide sequence.

The following description of the 5′ nucelase PCR nucleic acid amplification assay of the present invention is general in nature. It is intended only to assist the reader in understanding the novel features of the present invention. It is understood that nucleic acid amplification reactions are complex dynamic processes. However, for illustration purposes, the assay description will be described in discrete steps. In actuality, multiple processes are occurring simultaneously. Wherever possible the interrelationship between steps in the amplification processes will be brought to the reader's attention.

The 5′ nuclease assay of the present invention is initiated by mixing a PCR master mix containing primer, target oligonucleotide probes, optionally internal control oligonucleotides, internal control probes, Taq polymerase desoxynucleotide triphosphates (dNTP), cofactors, salts and buffer with the sample to form a reaction mixture. The reaction mixture is heated to denature target DNA present in the sample and then cooled to allow binding of the internal control probe to is complementary oligonucleotide. The reaction mixture is then optimized to facilitate primer binding to its complementary binding sites on either the target oligonucleotides and/or internal control oligonucleotides. Next, primer extension (amplification) is initiated as the Taq polymerase adds dNTPs to the 3′ ends of the primers bound to either target oligonucleotide (if present in the reaction mixture) and/or the internal control. During the amplification process, additional probes bind to the newly synthesized oligonucleotide as primer extension continues.

The Taq polymerase adds dNTP to the extending primer's 3′ end moving downtream towards the target or internal control oligonucleotide's 5′ end. As the Taq polymerase encounters probes previously bound to complementary sites on the oligonucleotide strands it exerts its 5′→3′ endonuclease activity and removes the bound probes one nucleotide at a time. The liberated probes are then detected indicating successful target or internal control oligonucleotide amplification. Detection of the internal control probe indicates that a successful amplification process has occurred. Consequently the operator can be assured that assay reaction conditions were appropriate and the PCR cocktail was working. Moreover, internal control probe detection indicates that the sample did not contain PCR amplification inhibitors. Consequently, negative results can be recorded with confidence knowing that if target oligonucleotide were present in the sample it too would have been amplified.

Probes can be detected in a variety of ways. In one embodiment of the present invention the probe's 5′ prime end nucleotide is conjugated to a fluorescent indicator molecule where as a fluorescent indicator quencher is bound to the probe's 3′ end. Fluorescent signal cannot be detected as long as the fluorescent indicator remains within a predetermined proximity of the quencher. However, as the 5′ nuclease removes probes from the target or internal control oligonucleotide one nucleotide at a time, the distance between the fluorescent indicator and its quencher molecule increases. Consequently, the fluorescent indicator is no longer quenched and its signal can be detected using fluorometric sensors or other methods known in the art. It will be apparent to persons having ordinary skill in molecular biology that not all probes will be cleaved from their complementary oligonucleotides as mononucleotides (that is, one nucleotide at a time), but rather may be removed as short oligonucleotides. Moreover, it is also possible for the entire probe to be cleaved simultaneously (strand displacement). Also, those skilled in the art will also recognize that many other indicator systems can be used to detect PCR amplification and 5′ nuclease activity. The preceding discussion was intended merely as an example and should not be construed as a limitation.

As previously explained, 5′ nuclease assay internal controls consist of oligonucleotides having primer-binding sites and complementary labeled oligonucleotide probes. The internal control oligonucleotide must be sufficiently different from the target oligonucleotide so that probes directed against the target do not bind to the internal standard. However, these differences cannot be so great that internal control oligonucleotide amplification does not reflect target oligonucleotide amplification. For example, the nucleotide composition of an oligonucleotide determines its annealing and denaturing properties. Oligonucleotides that are guanine (G) and cytosine (C) rich will have greater thermal stability than oligonucleotides with lower GC content. As a result, GC rich oligonucleotides have higher denaturing (melting) temperatures. Moreover, the probe's nucleotide base composition can also dictate how, and where the 5′ nuclease cleaves the probe from its complementary oligonucleotide strand. Probes having GC rich regions tend to be cleaved after the first or second nucleotide, where as adenine (A) and thymine (T) rich probes tend to be cleaved after the fifth or sixth nucleotide.

An internal control is used to detect nucleic acid amplification assay corruption, and to verify assay performance. Therefore, assay factors affecting target nucleic acid amplification and detection must similarly affect the internal control. Consequently, it is imperative that the internal control chemically and physically mimic the target oligonucleotide and its complementary probe. However, assay specificity mandates that the target probe not bind to complementary sites on the internal control oligonucleotide. It this occurs, false positive results may be obtained. Therefore, careful consideration must be given to the design and construction of the internal control olignucleotide and complementary probe pair.

Calculations necessary to determine oligonucleotide/probe annealing temperatures, melting points (Tₘ) and 5′ nuclease cleavage characteristics are extremely complex and time consuming. Moreover, the oligonucleotide/probe pairs based on these calculations must be extensively tested to verify assay performance. Furthermore, test specificity requirements dictate that each target oligonucleotide amplification and detection assay must have a different internal control oligonucleotide and complementary probe. Consequently, methods for quickly and accurately determining suitable internal control nucleic acid sequences are needed.

The present invention provides internal controls having nucleic acid sequences that are the inverse of the target nucleic acid sequences. For example, assume that a 5′ nuclease PCR assay is designed to detect target DNA having the following sequence:

5′-ATTCCCGTCAGGTCCAATTCC-3′

Then the internal control would have the following sequence:

5′-CCTTMCCTGGACTGCCCTTA-3′

The target sequence and the internal control would both have the same relative AT and GC ratios and consequently have identical annealing temperatures and Tₘ. Moreover, the complementary probes for both the target oligonucleotide and the internal control would possess similar, if not identical 5′ nuclease cleavage characteristics.

The present invention also provides 5′ nuclease assays having excellent specificity. Probes designed to bind to complementary regions on the target oligonucleotide would not recognize their inverse sequence. Therefore, a target nucleotide probe binding to an internal control having a probe binding sequence that is the inverse of the target oligonucleotide probe binding sequence can only occur when the target oligonucleotide sequence is a palindrome. Palindrome nucleic acid sequences are extremely rare and can be entirely avoided during the target nucleic acid sequence selection process.

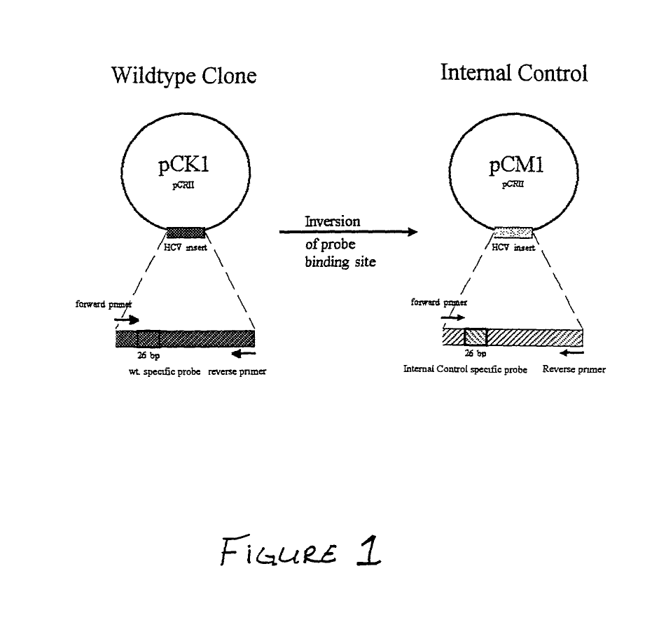

The internal control oligonucleotide sequences and complementary probes of the present invention can be prepared using techniques known to those skilled in the art. FIG. 1 depicts a method of using the wild type HCV plasmid pCK1 to prepare the internal control plasmid pCMI having an inverted wild type HCV probe binding site. Non-limiting examples of suitable methods include chemical syntheses, DNA replication, reverse transcription, and recombinant DNA techniques. The probes made in accordance with the teachings of the present invention may be labeled using any number of different techniques, including but not limited to enzymes, enzyme substrates, radioactive atoms, fluorescent dyes, chromophores, chemiluminescent materials, magnetic and paramagnetic particles, antibodies and other ligands. Detection methods appropriate for the label selected include spectroscopic, photochemical, biochemical and/or immunochemical means.

In one embodiment of the present invention only the internal control probe is labeled, in another embodiment the target oligonucleotide probe is exclusively labeled. In yet another embodiment of the present invention both probes are labeled. The labels may be the same or different. In one embodiment of the present invention the internal control probe has a fluorescent label and the target oligonucleotide probe has a radioactive label. It is understood by those skilled n the art that any number of possible combinations of labeled, unlabeled and multi-labeled probes can exist and that any such combinations can be practiced without departing from the spirit of the present invention and are considered to be part of the present invention.

One of the most important applications for the 5′ nuclease assays of the present invention is in the detection of blood borne pathogens in blood and blood products. Donated blood samples are screen for infectious agents and blood borne pathogens using antibody assays; however, even the most sensitive antibody assays cannot detect blood borne pathogens such as HCV or HIV prior to seroconversion. Generally, seroconversion occurs within 60 days in the majority of infected individuals. However, in immunologically impaired individuals, seroconvertion can be delayed up to one year or more. Consequently, HCV contaminated blood continues to enter the worlds blood supply at an alarming rate and is responsible for transfusion acquired HCV infection in approximately 9.7 persons per million transfusions. However, this transmission acquired infection rate could be cut to less than 3 persons per million transfusions if blood donations were screened using systems that can detect blood borne pathogens prior to seroconversion. The nucleic acid amplification and detection systems of the present invention provide rapid, sensitive and specific assays capable of pre-seroconvertion detection of blood borne pathogens including HCV.

The demand for highly specific blood products in acute clinical situations demands blood borne pathogen detection assays that are fast, robust, reliable and not prone to false positive or false negative results. The 5′ nuclease assay of the present invention offers these and other features. In one embodiment of the present invention a 5′ nuclease assay is provided that utilizes a closed system having both the target oligonucleotide amplification and detection step performed simultaneously using fluorescently labeled probes. Moreover, the internal control of the present invention is incorporated into this assay significantly reducing false negatives.

The following detailed example depicts how the present invention can be applied to the detection of HCV in human blood products including plasma. It is not indented as a limitation, but is offered to illustrate the advance the present invention represents over the current state-of-the art.

### EXAMPLE

### Development and validation of an HCV-RNA-PCR Assay

### Using 5′Nuclease Assay Technology

I. Methods and Materials

A. Samples and Controls

Assay sensitivity, specificity and reproducibility was validated using WHO-international HCV standards and a quantified positive HCV standard calibrated against the WHO standards. Genotype recognition and differentiation was validated using an HCV-genotype panel (German reference center for HCV, Essen, Germany). A sample test panel of approximately 150 expired blood donation samples and synthetic HCV reactive samples were prepared using negative plasma samples spiked with HCV positive material.

B. Sample preparation and distribution

Samples were collected in 9 ml EDTA-tubes (Sarstedt, Nümbrecht, Germany). EDTA-plasma was separated from cells within 18 hours after collection and used for extraction. Each test sample was dispensed as follows: two individual 800 μL aliquots were reserved frozen; 100 μL to 300 μL of each sample was added to a plasma pool; 700 μL aliquots of samples intended for platelet apheresis concentrates were stored individually. An additional 1.6 mL of the original EDTA plasma was reserved in 3 ml Cryovials (Simport, Quebec, Canada) and stored refrigerated. Unique identifying bar code labels were provided for each sample and aliquots thereof. Sample dispensing was conducted using an automatic multipipetter (Genesis 150/8, TECAN, Crailsheim, Germany).

C. Antibody Screening

Blood donations were routinely screened for anti-HCV antibodies using the Ortho HCV 3.0 ELISA test (Ortho-Clinical Diagnostics, Neckargmünd, Germany).

D. RNA Isolation

HCV RNA extraction was performed using Qiamp viral RNA kit, (QIAGEN, Hilden, Germany) according to the manufacturer's instruction. Briefly, 560 μL of AVL-buffer/carrier-RNA are added to 140 μL of each plasma pool. After incubation for 10 minutes at 56° C. on a heated shaker, 560 μL of absolute ethanol was added. Next, 630 μL of sample is added to spin tubes containing silica membranes that bind the viral RNA. After washing, the viral RNA is eluted in 50 μL of purified water. This procedure is performed in duplicate. The extracted RNA is now ready RT/PCR testing or can be preserved by storing at −80° C.

E. External Standard CK1 used for HCV quantitation

A quantitative standard, CK1 is used to permit simultaneous quantitation and detection of HCV RNA. The CK1 standard an in vitro-transcript derived from the plasmid pCK1. Plasmid pCK1 was cloned by introducing 559 bp of the HCV-wild type (bases 43 to 601-genebank sequence HPCCGM) into plasmid pCRII (Invitrogen, Groningen, NL). Amplification and detection is performed with primer CT1.f (forward) and CT1.r (reverse) and with 6-carboxy-fluorescein (6-FAM)-labeled target probe CT1.p.

F. Internal control Probe CM1

The internal control CM1 was produced using in vitro-transcription of the plasmid pCM1, which carries the same HCV-sequence as pCK1 with the exception that the binding-site for the internal control probe is inverted. The internal control CM1 probe was labeled using tetra-chloro-carboxy-fluorescein (TET) which was used for detection. Amplification was performed using primers CT1.f and CT1.r.

G. Sequences of primers and probes

| | | |
|---|---|---|
| SEQ. ID 1 | 5′ CCCTGTGAGGAACTACTGTCTTCA 3′ | (Forward primer) |
| SEQ. ID 2 | 5′ ACTCACCGGTTCCGCAGA 3′ | (Reverse Primer) |
| SEQ. ID 3 | 5′ 6-FAM-TGGCGTTAGTATGAGTGTCGTGCAGC-TAMRA 3′ | (Target probe) |
| SEQ. ID 4 | 5′ TET-CGACGTGCTGTGAGTATGATTGCGGT-TAMRA 3′ | (Internal control Probe) |

H. 5′ nuclease RT-PCR amplification and detection assay

10 μL of the extracted sample RNA were mixed with 40 μL of PCR master mix (Taqman-EZ-RT-RNA-kit® (Roche Molecular Systems, Branchburg Township, N.J.) components (5xEZ-buffer, 25 mM MnAc₂ 10 mM dNTP-only dUTP 20 mM, AmpErase, 5 U of rTth-polymerase; Perkin Elmer, Weiterstadt, Germany) and 10 pmol of forward primer, fluorescent probes for wild type HCV sequence and CM1 and 50 pmol of reverse primer. For a complete control of the reverse transcription and amplification, 1000 copies of the internal control CM1 (Baxter AG, Vienna, Austria) was added to each reaction tube.

Reverse transcription and PCR are performed as a single step reaction with rTth-polymerase (Perkin Elmer, Weiterstadt, Germany), which has a reverse transcriptase activity in the presence of manganese-ions. Two primers are used for PCR and one sequence specific probe each for detection of wild type HCV oligonucleotide and the internal oligonucleotide.

The internal control probe (CM1) and target probe (CT1p) were labeled with different reporter dyes (Perkin Elmer) at their 5′ end and the same quencher molecule consisting of 6-carboxy-N,N,N′,N′-tetrachlorofluorescein (TAMRA, Perkin Elmer) was added to the 3′ end of each probe. A two minute step is performed at 50° during thermal cycling to activate the uracil-N-glycolase (UNG) activity of AmpErase to prevent potential contamination carry-over. Reverse transcription is performed at 59° C. for 20 min. deactivation of UNG and denaturing 5 min at 95° C. 45 cycles are used for amplification with a denaturing step (94° C., 20 s) and an annealing/extension step (57° C., 1 min).

While the probes are intact, fluorescence of the reporter dye is quenched. If hybridization of the probes to the DNA occurs, the probes are degraded through the nuclease activity of the polymerase, reporter and quencher dyes are separated and the emission spectrum of the label is detectable. The amplicons are amplified exponentially resulting in a measurable increase of the fluorescence. The 5′ nuclease PCR technology of the present invention permits real-time observation of the DNA amplification. The cycle at which the fluorescence rises higher than the background signal is called the C_{T}-value (threshold cycle) and is proportional to the concentration of the viral RNA in the extracted sample. This allows target signal quantitation when a standard curve is measured in parallel with the external standard CK1.

II. Results and Discussion

The 5′ nuclease PCR technology of the present invention was implemented in three steps. During the first step the detection limit and the reproducibility of the method was determined. The second step was necessary to test the reliability and robustness of the experimental set up with blinded panels (spiked with positive HCV samples). In the third and final step 100 individual blood samples were screened to assess assay specificity under simulated routine conditions.

A. HCV Positive Control

A calibrated HCV positive plasma sample was used for positive control purposes throughout this study. The positive control was calibrated against the WHO international standard (Lot.-Nr.: 96/790, NIBSC, South Mimms, UK). The WHO international standards prepared as describe in the assay sensitivity results immediately below. Each diluted WHO standard and HCV positive control plasma were pre-diluted 1:100, extracted in parallel and analyzed with the 5′ nuclease PCR technology of the present invention. The HCV positive control plasma demonstrated a mean viral load of 1.4×10⁶ IU/ml when compared to the WHO international standard. For routine screening the HCV positive control was used at a concentration of 480 IU/ml.

B. Sensitivity

Assay sensitivity was determined using WHO-international standard (Lot.-Nr.:96/790, NIBSC, South Mimms, UK). Standards were tested in eight dilution series (half logarithmic) starting with a concentration of 5000 IU/ml. For each series eight replicates of each dilution were prepared. Each dilution series was tested a total of three times on three different days using different personnel. A total of 24 extractions and PCR assays were performed on each dilution. Table 1 depicts the results of this analysis. The first column depicts HCV-RNA present in each WHO standard dilution expressed as International Units (IU) per mL. Column two depicts the calculated counts of fluorescent HCV probe detected per second (cps) per mL of diluted WHO standard (cut-off values for positive results). Column three depicts the percentage of WHO standard dilution that tested positive (had cps/mL equal to or greater than the calculated values) for each HCV-RNA concentration. The results presented in Table 1 were analyzed with the logit method (FIG. 2). A 95% detection limit of 280 IU/ml corresponding to 8 IU directly in the reaction tube is reported.

TABLE 1
Quantitative Sensitivity of the 5′ nucleotide HCV PCR Assay of the
Present Invention using an WHO HCV International Standard

| HCV-RNA in WHO | Calculated | Percentage of Stanard |
|---|---|---|
| Standard | HCV-RNA | Positive results |
| (IU/ml) | (cps/ml) | (%) |
| 500 | 5510 | 100 |
| 165 | 597 | 87.5 |
| 54 | 313 | 45.8 |
| 18 | 70.5 | 8.3 |
| 6 | 209 | 16.7 |
| 0 | 0 | 0 |

C. Specificity

The 5═ nuclease PCR assay of the present invention uses sequence specific probes to detect amplified HCV genome. Therefore, the detection of non-specific primer amplification is unlikely. Furthermore, cross-contamination during sample preparation, assay set up amplification is minimized due to the use of different laboratories and dedicated equipment for each assay step. One hundred plasma samples from random blood donation were tested using the 5′ nuclease PCR assay of the present invention to test assay specificity. No false positive results could be detected.

Reproducibility

Quantitative reproducibility was assessed using eight replicate samples of the HCV positive control calibrated against the WHO standard as explained above. The eight replicate panel were assayed using the 5′ nuclease PCR assay of the present invention on three successive runs. Results are provided in Table 2. Assay reproducibility and precision is expressed in terms of coefficient of variation (standard deviation/mean). The intra-assy coefficient of variation (CV) varied from 31.2 to 34.8%. The inter-assay coefficient of variation was 39%.

TABLE 2
HCV Quantitative 5′ Nuclease PCR Assay Reproducibility

| | Panel Member | Panel Member | Panel Member |
|---|---|---|---|
| | I* | II* | III* |
| 1 | 3386 | 2823 | 2068 |
| 2 | 1300 | 3067 | 2576 |
| 3 | 1589 | 2040 | 3033 |
| 4 | 1710 | 4898 | 4120 |
| 5 | 2437 | 4265 | 5215 |
| 6 | 1864 | 1906 | 3276 |
| 7 | 2335 | 4057 | 5178 |
| 8 | 2636 | | 3972 |
| Mean | 2157 | 3294 | 3680 |
| SD | ±675 | ±1145 | ±1151 |
| CV | ±31.2% | ±34.8% | ±31.3% |

*ResuIts are expressed in cps/ml (in-house units).

E. Assay False Positive and False Negative Results

As discussed in detail above, it is desirable to incorporate internal controls into the 5′ nuclease PCR assay of the present invention in order to validate amplification and assay integrity. In the HCV 5′ nuclease assay of the present invention, an internal control designated CM1 was incorporated into to each PCR assay to minimize false negative result reporting. During routine use of the HCV 5′ nuclease assay of the present invention the internal control detected amplification failures in approximately 1.2% of the samples. All samples that failed to amplify were re-extracted and tested again using the HCV 5′ nuclease assay of the present invention. All false negative samples resulted in valid results in the repeat assay.

False positive results primarily result from sample contamination during processing, extraction, assay set up or post amplification manipulations. Performing each assay step using a separate, isolated laboratory having dedicated equipment and materials can significantly reduce sample contamination. Moreover, the 5′ nuclease PCR assay the present invention eliminated the need for post processing manipulations due to the ability to detect specific target oligonucleotides during amplification. Poor specificity of the detection probe or target sequence selected for amplification can also contribute to false positive reactions using nucleic acid amplification techniques. However, the primers and probes of the HCV 5′ nuclease assay of the present invention demonstrated excellent specificity with a minimum of false positive results.

The preceding Example provides a specific and sensitive qualitative HCV 5′ nuclease PCR assay that incorporates an internal control made in accordance with the teachings of the present invention. However, the methods of the present invention can be used to provide any 5′ nuclease with a specific, sensitive internal control that closely mimics the chemical and physical properties of the target oligonucleotide/detection probe pair. This is accomplished by inverting the target probe binding site oligonucleotide sequence and preparing an internal standard oligonucleotide using the inverted sequence. The present invention provides a rapid and accurate method for preparing 5′ nuclease PCR assay internal controls when compared to conventional methods for internal control sequence selection.

All patent, patent applications, and technical references, including manufacture instructions and references manuals, identified in this patent are hereby incorporated by reference in their entirety.

In the foregoing description of the present invention, preferred exemplary embodiments of the invention have been disclosed. Particular reference has been given to internal controls for quantitative HCV 5′ nuclease PCR assays. It is to be understood by those skilled in the art that other equivalent methods and internal controls are within the scope of the present invention. Accordingly, the present invention is not limited to the particular exemplary compositions that have been illustrated and described in detail herein.

What is claimed is:

1. A method for preparing an internal control for a 5′ nuclease polymerase chain reaction (PCR) assay comprising:
determining a nucleic acid sequence for a target oligonucleotide probe binding site;
inverting said nucleic acid sequence of said target oligonucleotide probe binding site; constructing an internal control oligonucleotide having said inverted target oligonucleotide probe binding site integrated therein;
constructing an internal control probe having a nucleic acid sequence complementary to said inverted target oligonucleotide probe binding site, wherein said internal control probe hybridizes with said inverted target nucleic acid probe binding site sequence integrated into said internal oligonucleotide control but not with said nucleic acid sequence of said target oligonucleotide probe binding site.

2. The method for preparing an internal control for a 5′ nuclease PCR assay of claim 1 wherein said internal control probe has a detectable label.

3. The method for preparing an internal control for a 5′ nuclease PCR assay of claim 2 wherein said detectable label is selected from the group consisting of fluorescent labels, radioactive labels, antibody labels, chemiluminescent labels, paramagnetic labels, enzymes and enzyme substrates.

4. The method for preparing an internal control for a 5′ nuclease PCR assay of claim 1 wherein said target oligonucleotide probe and said internal control probe have detectable labels.

5. The method for preparing an internal control for a 5′ nuclease PCR assay of claim 4 wherein said target oligonucleotide detectable label is different than said internal control probe detectable label.

6. The method for preparing an internal control for a 5′ nuclease PCR assay of claim 1 wherein said target oligonucleotide and said internal control oligonucleotide are amplified by the same primers.

7. A 5′ nuclease PCR assay having an internal control wherein said internal control comprises:
an oligonucleotide having at least part of its nucleic acid sequence an inverse of a target oligonucleotide probe binding site nucleic acid sequence;
an internal control probe having a nucleic acid sequence complementary to said inverted target oligonucleotide probe binding site, wherein said internal control probe hybridizes with said inverted target nucleic acid probe binding site sequence but not with said target oligonucleotide probe binding site nucleic acid sequence.

8. The 5′ nuclease PCR assay having an internal control of claim 7 wherein said internal control probe has a detectable label.

9. The 5′ nuclease PCR assay having an internal control of claim 8 wherein said detectable label is selected from the group consisting of fluorescent labels, radioactive labels, antibody labels, chemiluminescent labels, paramagnetic labels, enzymes and enzyme substrates.

10. The 5′ nuclease PCR assay having an internal control of claim 7 wherein said target oligonucleotide probe and said internal control probe have detectable labels.

11. The 5′ nuclease PCR assay having an internal control of claim 10 wherein said target oligonucleotide detectable label is different than said internal control probe detectable label.

12. The 5′ nuclease PCR assay having an internal control of claim 7 wherein said target oligonucleotide and said internal control oligonucleotide are amplified by the same primers.

13. The 5′ nuclease PCR assay having an internal control of claim 7 wherein said 5′ nuclease PCR assay is for the detection of pathogens.

14. The 5′ nuclease PCR assay having an internal control of claim 13 wherein said pathogens are selected from the group consisting of human immunodeficiency viruses (HIV), hepatitis C virus (HCV), hepatitis B virus (HBV), human parvovirus, and hepatitis A virus.

15. An HCV 5′ nuclease PCR assay comprising:
a first probe having a first detectable label, said first probe having a nucleic acid sequence complementary to a target HCV oligonucleotide probe binding sequence;
a 5′ nuclease enzyme;
a second probe having a second detectable label, said second probe having a nucleic acid sequence complementary to an internal standard oligonucleotide probe binding sequence, said internal standar oligonucleotide probe binding sequence being the inverse of said target HCV oligonucle tide probe binding sequence;
at least one primer complementary to primer binding sites on said target HCV nucleotide and said internal standard oligonucleotide.
at least one primer complementary to primer binding sites on said target HCV nucleotide

16. The HCV 5′ nuclease PCR assay of claim 15 wherein said first and said second detectable labels are selected from the group consisting of fluorescent labels, radioactive labels, antibody labels, chemiluminescent labels, paramagnetic labels, enzymes and enzyme substrates.

17. The HCV 5′ nuclease PCR assay of claim 15 wherein said first probe and said second probe have different detectable labels.

18. The HCV 5′ nuclease PCR assay of claim 15 wherein said first probe's nucleic acid sequence is SEQ. ID 3.

19. The HCV 5′ nuclease PCR assay of claim 15 wherein said second probe's nucleic acid sequence is SEQ. ID 4.

20. The HCV 5′ nuclease PCR assay of claim 15 wherein said HCV 5′ nuclease PCR assayis a quantitative assay.
